(19) United States
(12) Patent Application Publication
(10) Pub. No.: US 2009/0214484 A1
(43) Pub. Date: Aug. 27, 2009

(54) STEM CELL THERAPY FOR THE TREATMENT OF CENTRAL NERVOUS SYSTEM DISORDERS

(76) Inventor: NIKOLAY MIRONOV, Moscow (RU)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/036,192

(22) Filed: Feb. 22, 2008

(51) Int. Cl. A61K 35/12

(52) U.S. Cl. 424/93.7

(57) ABSTRACT

The invention provides a method for treating CNS disorders by administering a neural stem cell composition and a mesenchymal stem cell composition on opposing sides of the blood brain barrier. The neural stem cell composition is administered to the central nervous system, while the mesenchymal stem cell composition is administered to the circulatory system, such as by intravenous injection. The method finds use in the treatment of degenerative GNS disorders, as well as traumatic CNS disorders such as stroke and spinal cord injury.

### BACKGROUND OF THE INVENTION

GNS disorders encompass numerous afflictions such as acute brain injury (e.g. stroke, head injury, cerebral palsy), spinal cord injury, neurodegenerative diseases (e.g. Alzheimer's and Parkinson's), and a large number of CNS dysfunctions (e.g. depression, epilepsy, and schizophrenia). Degeneration in a brain region known as the basal ganglia can lead to diseases with various cognitive and motor symptoms, depending on the exact location.

Stroke is the leading cause of adult disability and the third cause of death worldwide. In the United States alone, a person has a stroke every 45, second's, which accounts for approximately 700,000 people per year. Stroke is the third leading cause of death in the U.S., and it can lead to severe, long-term disability. In fact, more than two-thirds of stroke survivors are left with significant sensory motor impairment.

Stroke is a type of cardiovascular disease, which affects the arteries leading to and within the brain. When a stroke occurs, part of the brain starts to die from lack of blood flow and the part of the body it controls is affected. Damage to the brain can cause loss of speech, vision, or movement in an arm or a leg, depending on the part of the brain that is affected. Treatments are available to minimize the potentially devastating effects of stroke, but to receive them one must recognize the warning signs and act quickly.

A stroke occurs when a blood vessel that carries oxygen and nutrients to the brain is either blocked by a clot or bursts. Clots that block an artery cause ischemic strokes, which account for about 70 to 80 percent of all strokes. Cerebral ischemia induced by stroke leads to rapid death of neurons and vascular structures in the supplied region of the brain.

Alzheimer's disease, there is a profound cellular degeneration of the forebrain and cerebral cortex. In the case of Parkinson's disease, degeneration is seen in the substantia nigra par compacts. This area normally sends dopaminergic connections to the striatum that are important in regulating movement. Dopamine is a catecholamine neurotransmitter that is particularly important in the control of movement. The great majority of brain dopamine is found in the striatum, and contained in neurons originating from a brain stem nucleus, the substantia nigra. The death of these cells, with a consequent loss of dopamine, is responsible for the symptoms of Parkinson's disease.

There is substantial evidence in both animal models and human patients that neural transplantation is a scientifically feasible and clinically promising approach to the treatment of neurodegenerative diseases and stroke as well as for repair of traumatic injuries to the brain and spinal cord. However, the administration of neural cells alone does not address the endothelialization that needs to occur in order to support the endogenous and transplanted cells with a blood supply. What is heeded therefore is a treatment therapy that addresses both the regeneration of damaged or lost neurons, and the regeneration of the endothelial framework for the damaged area.

### SUMMARY OF THE INVENTION

An objective of the invention is to provide a method for treating a central nervous system (CNS) disorder comprising administering on the neural side of the blood brain barrier (BBB) a composition having a therapeutically effective amount of neural stem cells, and administering on the circulatory side of the BBB a composition having a therapeutically effective amount of mesenchymal stem cells. The neural stem cell composition may be administered intrathecally, while the mesenchymal stem cell composition may be administered intravenously. The method may be used to treat a variety of CNS disorders including stroke, multiple sclerosis, cerebral palsy, Parkinson's disease, Alzheimer's disease, ischemic injury, and traumatic injury.

### BRIEF DESCRIPTION OF THE DRAWINGS

FIG. 1 shows a phase-contrast microscopy (PCM) image of a primary suspension of dissociated brain tissue cells from an embryo at 10 weeks of development.

FIGS. 2 and 3 show microscopic images of neuroepithelial cells after 15 days and 25 days in culture respectively.

FIG. 4 shows a microscopic image of neuroepithelial cells after after 5 passages.

FIG. 5 shows a pair of microscopic images of neuroepithelial cells showing positive staining for nestin.

FIG. 6 is a microscopic image of neuroepithelial cells with positive staining for and beta-tubulin III.

FIG. 7 is a microscopic image of neuroepithelial cells showing positive expression for glial fibrillary acidic protein (GFAP).

FIG. 8 is a microscopic image of a primary bone marrow cell suspension.

FIGS. 9 and 10 depict microscopic images of primary human bone marrow cell cultures at 4 and 10 days respectively.

FIG. 11 is a light microscopy image of human mesenchymal stem cells.

FIG. 12 is a microscopic image of human mesenchymal stem cell colonies after 10 days in culture and staining with an alcohol solution of 0.5% crystal violet.

FIGS. 13a-f are graphs showing the expression profiles of human mesenchymal stem cells for CD105, CD90, CD44, CD34, HLA-ABC and HLA-DR respectively.

### DEFINITIONS

As used herein, the phrases “according to the invention,” “according to the method of the invention,” other references to “the invention,” and “according to the methods disclosed herein” shall refer to, at a minimum, a method for treating a CNS disorder comprising (a) administering a neural stem cell composition to the neural side of a patient's blood brain barrier, and (b) administering a mesenchymal stem cell composition to the circulatory side of the patient's blood brain barrier.

A “central nervous system disorder,” or “CNS disorder,” refers to a condition or injury that impairs the normal function of the mammalian central nervous system, such as, for example, neurodegenerative disorders, traumatic injuries (to the brain or spinal cord) and CNS dysfunctions. Neurodegenerative CNS disorders are generally associated with a prolonged deterioration of CNS neural tissue including, but not limited to, Alzheimer's disease, Parkinson's disease, multiple sclerosis (MS), Huntington's disease, amyotrophic lateral sclerosis, cerebral palsy, Gaucher's disease, Tay-Sachs disease, Niemann Pick's disease, sphingomyelin lipidoses, and brain tumors. CNS disorders further include traumatic injuries, such as for example, hemorrhagic stroke, ischemic stroke, and mechanical injuries to the brain and spinal cord. The phrase “CNS disorder” further includes dysfunctions such as, for example, depression, epilepsy, and schizophrenia.

The term “spinal cord injury” refers to a condition occurring when a traumatic event damages cells within the spinal cord, or when the nerve tracts that relay signals,up and down the spinal cord are severed or otherwise injured. Some of the most common types of spinal cord injury include contusion and compression. Other types of injuries include, but are not limited to lacerations, and central cord syndrome.

Traumatic Brain Injury (TBI) is caused primarily by a traumatic blow to the head causing damage to the brain, often without penetrating the skull. The initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

The term “ischemia” refers to local anemia due to mechanical obstruction of the blood supply. “Ischemic” refers to a tissue that has been damaged by ischemia.

The term “stroke” refers to a condition wherein the blood flow to the brain stops or is restricted to the point of causing an impairment of neurological function. The term “stroke” includes ischemic stroke, which may be caused by an obstruction that blocks a blood vessel or artery in the brain, and hemorrhagic stroke which may be caused when a blood vessel in the brain ruptures and spills blood into the surrounding tissue.

The term “CNS ischemia,” as used herein, is intended to refer to the partial, or complete reduction of blood flow to one or more areas of the brain or spinal cord. The ischemia can be global, e.g. a generalized reduction in blood flow due to systemic hypotension, or focal, e.g. due to a disease in one or more cerebral arteries of localized trauma. The ischemia may be the result of stenosis or occlusion of a blood vessel, for example due to a thrombosis, an embolism,, or particle.

The term “neuronal damage,” or “neuronal injury,” as used herein is intended to refer to the:damage that occurs to any cell type (e.g. neurons, astrocytes, glia) in the CNS as a result of a CNS disorder or injury. For example, a lack of blood flow results in the death of cells by necrosis and/or apoptosis.

The term “stem cell” refers, to an undifferentiated cell which has the ability to both self-renew and undergo differentiation to form one or more specialized cell types. Stem cells have varying degrees of potency.

The term “precursor cell,” Or “tissue precursor cell,” refers to an undifferentiated cell that is committed to a specific developmental pathway. Precursor cells have limited proliferative ability and unlike stem cells, are incapable of self-maintenance. Precursor cells are the lineage-committed progeny of self-renewing stem cells. A “neuronal precursor,” which is an undifferentiated cell dedicated to the development of a neuron, is one example of a precursor cell.

“Pluripotent” or “pluripotency,” refers to the ability of a stem cell to form specialized cells belonging to the mesoderm, endoderm and ectoderm tissue lineages.

The term “multipotent,” or “multipotency” refers to the ability of a stem cell to form more than one cell type belonging to a single germ lineage (e.g. the endoderm or ectoderm or mesoderm). For example, a cell which has the ability to form chondrocytes, adipocytes and osteocytes is a multipotent mesenchymal cell.

The terms “neural stem cell,” and “neural progenitor cell,” are used interchangeably to refer to a stem cell which is capable of self renewal (i.e. self maintenance) and which has the ability to differentiate to form at least one type of neural cell (e.g. neurons, astrocytes and oligodendrocytes). Neural stem cell progeny may remain undifferentiated and retain the ability to self renew, or they may differentiate to form neural precursors which are committed to develop into a specific neural cell type. Neural stem cells have varying degrees of potency and include multipotent ectodermal cells, and pluripotent cells.

As used herein the phrase “neural cell” includes both nerve cells' (i.e.,, neurons, e.g., uni-, bi-, or multipolar neurons) and their precursors and glial cells (e.g., macroglia such as oligodendrocytes, Schwann cells, and astrocytes, or microglia) and their precursors. “Neural-potent,” or “neural-potency,” refers to the ability of a stem cell to differentiate into a cell having a neural cell phenotype.

As used herein, the phrase “mesenchymal stem cell (MSC)” or “mesenchymal cell,” refers to undifferentiated, self-renewing cells of mesodermal germ lineage which have the ability to divide and form one or more specialized cell types such as, for example, endothelium, muscle, adipose, connective tissue, bone, cartilaginous tissue, and the various cells of the immune system. Mesenchymal stem cells have varying degrees of potency ranging from multipotent stem cells, down to tissue precursor cells which are committed to forming a specific cell type. Mesenchymal stem cells may be derived from tissues including, but not limited to, bone marrow, blood, dermis, periosteum, peripheral blood, skin, hair root, muscle tissue, uterine endometrium, umbilical cord blood, and amniotic fluid.

The term “blood brain barrier” (BBB), refers to the selective barrier that separates the blood from the parenchyma of the central nervous system. This barrier is formed by a system of tight junctions between the capillary endothelial cells that separate the blood from the cerebrospinal fluid and the extracellular fluid of the brain and spinal cord. The “neural side” of the BBB refers to the parenchyma (i.e. neural compartment, or neural parenchyma), and its associated neural tissues, which are surrounded by the BBB. The “circulatory side” of the BBB refers to the circulatory spaces (circulatory system) in the body.

As used herein,, a “therapeutically effective amount” refers to the number of transplanted cells which are required to produce a therapeutic effect for the disorder which is being treated. For example, where the treatment is for Parkinsonism, transplantation of a therapeutically effective amount of cells will typically produce a reduction in the amount and/or severity of the symptoms associated with that disorder, e.g., rigidity, akinesia and gait disorder.

The terms “prenatal” and “fetal” refer to the period that precedes the birth of a fetus, beginning with the formation of a diploid zygote. Thus, in the context of the invention, tissues and their associated cells derived from a fetus prior to natural birth, or birth by cesarean section, are fetal (i.e. prenatal) tissues. Tissues obtained from mammalian tissue following the birth (e.g. live and still birth) of the mammal are adult tissues and cells derived therefrom are “adult cells.” Fetal tissue and fetal cells may be obtained from, for example, miscarried and aborted fetuses. The stem cells of the invention may be derived from fetal tissues, adult tissues, and combinations thereof.

A “heterogeneous cell population,” or a “mixed cell population” refers to diversity in a cell population defined by different phenotypic features or molecular signatures, such as heterogeneity of ligands, e.g., antigens, epitopes or receptors on the surface of the cells. The cells belonging to a mixed population of cells may vary in their level of plasticity, the germ lineage from which they are derived, as well as their genotype as the invention contemplates mixtures of cells derived from the tissues of different human subjects. Typically, a primary cell culture from a tissue will have, a mixed population of cells as the cells expanded from a tissue explant will vary in their levels of differentiation, and consequently, their cell marker expression profiles. As used herein, a “combination of cells,” or “combined cell population,” refers to mixed cell population obtained by combining two or more purified (e.g. clonal) celltypes. The invention further contemplates a “mixed cell population” that is obtained by combining one or more purified cell populations with a population of mixed cell types such as that obtained from a primary culture.

As used herein, “treating a host,” or “treatment,” includes prophylactic, palliative, and curative intervention in a disease process. Thus, the term “treatment” as used herein, typically refers to therapeutic methods for reducing or eliminating the symptoms of the particular disorder for which treatment is sought. The term “host,” as used herein, generally refers to any warm blooded mammal, such as humans, non-human primates, rodents, and the like, which is to be the recipient of the particular treatment. The terms “host,” “patient” and “subject” are used interchangeably.

The term “clone,” or “clonal cell,” refers to a single cell which is expanded to produce an isolated population of phenotypically similar cells (i.e. a “clonal cell population”).

The term “cell line” refers to one or more generations of cells which are derived from a clonal cell.

### DETAILED DESCRIPTION

The invention provides methods for the treatment of central nervous system (CNS) disorders. The methods rely on the administration of therapeutic compositions of neural stem cells and mesenchymal stem cells. The neural stem cell composition is administered (e.g. intracranially or intrathecally) to the neural parenchyma formed by the blood brain barrier, while the mesenchymal stem cell composition is administered intravenously. The method finds use in the treatment of a variety of CNS disorders including stroke, Parkinson's disease, Alzheimer's disease, and traumatic injury such as brain and spinal cord damage.

1. Neural Stem Cell Composition

Cells for making the neural stem cell composition of the invention may be derived from any source so long as the cells derived from that source provide a therapeutic effect when administered according to the methods disclosed herein. The composition and specific source of neural stem cells used for practicing the invention will of course depend upon the type of CNS disorder that is being treated.

Neural stem cells for use with the invention may be derived from a variety of tissue compartments. In some embodiments, the neural stem cells are derived from nervous tissue. These neural-derived stem cells self-renew and remain undifferentiated in culture, assume a neural morphology upon introduction to mitogens in vitro, or upon introduction to the nervous tissue of an animal, and produce a regenerative effect when administered according to the methods disclosed herein. Neural tissue for providing a suitable source of neural stem cells includes (i) the peripheral nervous system, such as for example, the nasal epithelium, pigmented epithelium, non-pigmented epithelium, and ciliary body, (ii) the spinal cord, (iii) all the regions of the brain, including but not limited to, the forebrain, basal forebrain (cholenergic neurons), cerebellum, telencephalon, mesencephalon, hippocampus, olfactory bulb, cortex (e.g., motor or somatosensory cortex), striatum, ventral mesencephalon (cells of the substantia nigra), and the locus ceruleus (neuroadrenaline cells of the central nervous system), and (iv) combinations thereof.

Instructions for deriving neural stem cells from nervous tissue are readily available in the art as shown by the following publications which are incorporated by reference: U.S. Pat. Nos. 5,750,376, 6,497,872, and 6,777,233; U.S. Pat. Nos. 5,196,315; 5,766,948, 5,968,829; 6,468,794, 6,638,763, 6,680,198, 6,767,738, 6,852,532, 6,897,061, 7,037,719; U.S. Patent Publication Nos. 20050112109, 20040048373, 20020039789, 20020039789, 20030095956, 20050118143,20060148083, 20050074880, 20020086422, 20040253719, 20050003531, 20050125848, 20050142569, 20060099192 and 20060134280.

Neural stem cells for treating CNS disorders according to the methods disclosed herein may also be derived from non-neural (e.g. non-ectodermal) tissue sources. For example, stem cells capable of forming functional neural cells for treatment of a CNS disorder may be derived from mesenchymal stem cells. In some embodiments, this source of mesenchymal cells is the bone marrow. Such cells, in their undifferentiated state, assume a neural phenotype under in vitro conditions, or when introduced to the neural tissue of an animal. Amniotic fluid is another source of cells which can be differentiated into neural precursors. Instructions for deriving neural-potent bone marrow stem cells for use with the invention may be obtained from the following publication, which are incorporated by reference: U.S. Pat. Nos. 6,673,606 and 7,015,037; U.S. Patent Publication Nos. 20020164794, 20030003090, 20030039639, 20030059414, 20030203484, 20040151701, 20040208858, 20050282276, 20050249708, 20060105457, 20060177928; and Mareschi et al. Exp Hematol. November 2006;34(11): 1563-72. In other embodiments, neural-potent mesenchymal cells are derived from umbilical cord blood. Suitable umbilical cord-derived cells, and their methods of isolation, are disclosed in U.S. Patent Publication Nos. 20020028510, 20050249708, 20040115804, 20050142118 and 20050074435, the disclosures of which are incorporated by reference. Neural-potent mesenchymal cells may also be derived from the scalp (i.e. skin) (see e.g. U.S. Patent Publication Nos. 20030003574, 20040253718 and 20040033597; and Shih et al. Stem Cells August 2005;23(7) 1012-1020), the peripheral blood (see e.g. U.S. Patent Publication Nos. 20040136973 and 20050221483), the placenta (see e.g. U.S. Patent Publication Nos. 20050089513 and 20060030039) and the amniotic layer (see e.g. U.S. Patent Publication No. 20030044977).

The neural stem cell composition for use with the inventive method may be made using purified or non-purified cells, as well as combinations of purified and non-purified cells. Non-purified compositions of neural stem cells may be obtained in a number of ways. In some embodiments, the neural stem cell composition is made by combining separate, purified (i.e. isolated) neural stem cell populations. In other embodiments, the neural stem cell composition is obtained by culturing a mixed population of cells, such as a primary culture obtained from a tissue explant and expanded cell populations obtained therefrom. In still other embodiments, a non-purified composition of neural stem cells is obtained by combining one or more purified cell compositions, with a composition of mixed cell types such as a primary cell culture. Typically, primary cell cultures contain a mixture of cells as a variety of cells are able to grow in culture after being collected from an animal. Thus, primary cultures generally contain a combination of the different cell types which are able to proliferate in vivo. These cell types may have varying phenotypes (e.g. cellular markers) and varying levels of differentiation.

When the method is practiced using a primary culture of neural stem cells, the method generally involves the removal of a nervous tissue from an animal, disaggregation of the neural cells, within the sample, and expansion of the cells in a suitable media under appropriate in vitro conditions. In general, three types of cultures can be produced, enriched either in neurons, astrocytes, or oligodendrocytes. Methods for producing primary cultures of neural stem cells are widely available in the art. One such method is disclosed in U.S. Pat. No. 5,753,491, which describes the preparation of a neural stem cell composition from fetal neural tissue. In general, this process involves the collection of fetal brain tissue from fetuses between 9-11 weeks of gestational age (7-9 weeks postconception). Following extraction, brain tissue is disassociated to produce a cell suspension which is subsequently plated on culture dishes and expanded under suitable conditions. Although the preparation of human fetal neural tissue is specifically called out here, one skilled in the art will appreciate that fetal neural stem cells may also be derived from both human and non-human post-natal nervous tissue. The teachings of U.S. Pat. No. 5,753,491, and all other publications referred to in this publication are incorporated by reference in their entirety.

Other methods suitable for producing a primary culture of neural cells are readily available in the art. The following publications, which are incorporated by reference, provide the teachings necessary to enable one skilled in the art to prepare a primary culture of neural stem cells for use with the invention: U.S. Pat. Nos. 5,750,376, 6,497,872, and 6,777,233; U.S. Patent Publication Nos. 20050112109, 20040048373, 20020039789, 20020039789, 20030095956, 20050118143, 20060148083, and 20050074880; Isolation, Characterization and Use of Stem Cells from the CNS, 18 Ann. Rev. Neurosci. 159-92 (1995); M. Marvin & R. McKay, Multipotential Stem Cells in the Vertebrate CNS, 3 Semin. Cell. Biol. 401-11 (1992); R. P. Skoff, The Lineages of Neuroglial Cells, 2 The Neuroscientist 335-44 (1996). A. A. Davis & S. Temple, A Self-Renewing Multipotential Stem Cell in Embryonic Rat Cerebral Cortex, 362 Nature 363-72 (1994); A. G. Gritti et al., Multipotential Stem Cells from the Adult Mouse Brain Proliferate and Self-Renew in Response to Basic Fibroblast Growth Factor, 16 J. Neurosci. 1091-1100 (1996); B. A. Reynolds et al., A Multipotent EGF-Responsive Striatal Embryonic Progenitor Cell Produces Neurons and Astrocytes, 12 J. Neurosci. 4565-74 (1992); B. A, Reynolds & S. Weiss, Clonal and Population Analyses Demonstrate that an EGF-Responsive Mammalian Embryonic CNS Precursor is a Stem Cell, 175 Developmental Biol. 1-13 (1996); Cattaneo et al., Mol. Brain Res., 42, pp, 161-66 (1996); and B. P. Williams et al., The Generation of Neurons and Oligodendrocytes from a Common Precursor Cell, 7 Neuron 685-93 (1991).

Although fetal neural stem cell compositions are called out above, the inventive method of treating CNS disorders may also be practiced with compositions derived from adult neural tissue. Regenerative stem cells capable of correcting the symptoms of a variety of CNS disorders populate the adult animal brain. These cells remain undifferentiated in culture and are self-renewing under appropriate conditions. Upon the introduction of mitogenic factors, or the implantation in the nervous tissue of an animal, these cells assume a functional neural phenotype. Adult neural stem cells are also capable of ameliorating the effects of a variety of CNS disorders when administered according to the methods of the invention. Adult neural stem cells suitable for treating CNS disorders, and methods of deriving them, are taught in the following publications, the disclosures of which are incorporated by reference: U.S. Pat. Nos. 5,356,807, 5,851,832, 6,638,763 and 6,812,027; and U.S. Patent Publication Nos. 20030049234, 20030095956, 20030118566, 20040253719, 20050112109 and 20050118143.

Neural stem cells for use with the invention may be derived from human heterologous sources including fetal tissue following elective abortion, or from a post-natal, juvenile or adult organ donor. Autologous neural tissue can be obtained by biopsy, or from patients undergoing neurosurgery in which neural tissue is removed, for example, during epilepsy surgery, temporal lobectomies and hippocampalectomies. Neural stem cells have been isolated from a variety of adult CNS ventricular regions, including the frontal lobe, conus medullaris, thoracic spinal cord, brain stem, arid hypothalamus, and proliferated in vitro using the methods detailed herein. In each of these cases, the neural stem cell exhibits self-maintenance and generates a large number of progeny which include neurons, astrocytes and oligodendrocytes.

In addition to the use of primary cultures of neural stem cells, the method of the invention further contemplates compositions of purified neural stem cells for the treatment of central nervous system disorders. In the context of the invention, a cell composition is “purified,” or “isolated,” if the cells in the composition are essentially free from cells of a different type. A composition of cells is considered “purified,” or “substantially purified,” if it contains at least about 75%, at least about 80%, at least about 85%, at least about 90%, at least about 95% or at least about 100% of a desired cell type. Neural stem cells for use with the invention may be purified according to methods well known in the art, such as for example, FACS, magnetic sorting, serial passaging, cloning, and affinity chromatography. Such neural stem cells may be purified from a tissue explant or a mixed population of cells grown in culture. Suitable purified cells for practicing the invention, and the methods for making them, are disclosed in the following publications, the disclosures of which are incorporated by reference: U.S. Pat. Nos. 5,196,315, 5,766,948, 5,968,829, 6,468,794, 6,638,763, 6,680,198, 6,767,738, 6,852,532, 6,897,061 and 7,037,719; and U.S. Patent Publication Nos. 20020086422, 20040253719, 20050003531, 20050125848, 20050142569 and 20060099192.

In terms of the components of the neural stem cell composition, one skilled in the art will appreciate that different CNS disorders will require different neural stem cell compositions. For example, when the CNS disorder is a stroke, the method of the invention can optionally be practiced by administering a neural stem cell composition that is derived from fetal forebrain tissue (see Example 1). Other CNS disorders will require different neural stem cells which are capable of regenerating or augmenting the function of the neural tissue that has been compromised. For example, the treatment of Parkinson's disease according to the method of the invention may be practiced using a neural stem cell composition that is derived from cells capable of forming dopaminergic neurons (e.g. cells derived from mesencephalic tissue containing dopamine cells; see Freed et al. N Engl J Med 327:1549-1555 (1992); and Spencer et al. N Engl J Med 327:1541-1548 (1992); and Widheret al. N Engl J Med 327:1556-1563 (1992), each incorporated by reference).

The neural stem cells of the invention may be expanded using any method which produces a viable stem cell population capable of alleviating the effects of a CNS disorder when administered according to the invention. Fortunately, the culture of neural stem cells is an advanced art and the ordinary artisan has a wide variety of options for culturing neural stem cells for use with the inventive method. For example, U.S. Pat. No. 6,777,233 (incorporated by reference) teaches a method for the proliferation of neural stem cells using a medium containing leukemia inhibitory factor (“LIF”) which markedly and unexpectedly increases the rate of proliferation of the cells. This method offers the additional advantage Of enriching the neural cell cultures for “neurospheres” which are neuron-dedicated progenitor cells. Other suitable methods for culturing neural stem cells rely on a serum-free media, such as that disclosed in U.S. Pat. No. 6,020,197 which is incorporated by reference. This culture method provides a “perpetualized” population of neural stem cells which is capable of an indefinite number of divisions without requiring immortalization of the cells using exogenous DNA. Other methods for culturing the neural stem cells of the invention include, culturing the cells in serum-free media containing epidermal growth factor (“EGF”) or an analog of EGF, such as amphiregulin or transforming growth factor alpha (“TGF-.alpha.”), as the mitogen for proliferation (see e.g., WO 93/01275, WO 94/16718, both incorporated herein by reference). Still other methods for culturing neural stem cells are disclosed in the following publications, the teachings of which are incorporated by reference: WO 93/01275; WO 94/09119; WO 94/10292; Cattaneo et al., Mol. Brain Res., 42, pp. 161-66 (1996); U.S. Pat. Nos. 5,750,376 and 5,851,832, to Weiss et al.: U.S. Pat. No. 5,753,506, to Johe; U.S. Pat. No. 5,968,829 to Carpenter; Weiss et al., 19 Trends Neurosci. 387-93 (1996); Reynolds et al., 12 J. Neurosci. 4565-74 (1992); Reynolds & Weiss, 255 Science 1707-10 (1992); and Reynolds & Weiss, 175 Dev. Biol. 1-13 (1996) (all incorporated herein by reference).

In some embodiments of the invention, the neural stem cell composition is derived from neural stem cells which have been transfected with an exogenous polynucleotide. The neural stem cells may be transfected with any polynucleotide that encodes a protein that is beneficial to the CNS disorder that the invention is being used to treat. This may involve, for example, transfecting a neural stem cell to express a protein (e.g. enzyme) that deficient as a result of a particular CNS disorder. Such cells, when transplanted to a subject, will then produce a therapeutically effective amount of the deficient protein thereby improving the symptoms of the CNS disorder (see e.g. Rosenberg, et al., “Grafting genetically modified cells to the damaged brain: Restorative effects of NGF Expression,” Science 242:1575-1578, (1988).

In some embodiments, the neural stem cell composition is optionally replaced, or supplemented with, a population of NT2N cells. NT2N cells are derived from a human tefatocarcinoma cell line, the progeny of which are committed to the development of neurons. NT2N cells exhibit properties of CNS neurons, i.e. they express almost exclusively the 695 amino acid long amyloid precursor protein (APP), produce and secrete the .beta.-amyloid or A4 (.beta./A4) peptide found in Alzheimer's disease amyloid plaques and bear glutamate receptor channels on their cell surface. Methods for deriving NT2N cells are disclosed in the following publications which are incorporated by reference: Abramham, I. et al. 1991 J. Neurosci. Res. 28:29 39; Andrews, P. W., et al. 1981 Tissue Antigens 17:493 500; Andrews, P. W. et al 1984. 1984 Lab. Invest, 50 147 162; Andrews, P. W. 1987, Devel. Biol. 103:285 293; Kleppner, S. R., et al 1992 Soc. Neurosci. Abst. 18:782; Lee, V. M.-Y. and P. W. Andrews 1986 J. Neurosci. 6:514 521; Younkin, D. P. et al. 1993 Proc. Natl. Acad. Sci. U.S.A. 90:2174 2178; U.S. Pat. No. 5,175,103).

2. Mesenchymal Stem Cell Composition

An aspect of the invention relates to the administration of a composition of mesenchymal stem cells. In this regard, the invention may be practiced with any population(s) of mesenchymal stem cell(s) capable of producing a therapeutic benefit to a subject suffering from a CNS disorder when the composition is administered according to the invention.

In the context of the invention, mesenchymal stem cells are self-renewing cells which have the ability to form at least one cell type of mesodermal germ lineage (e.g. endothelium, muscle, adipose, connective tissue, bone* cartilaginous tissue, and the various cells of the immune system). These stem cells have varying degrees of potency ranging from multipotent stem cells, down to tissue precursor cells (e.g. endothelial precursor cells).

Mesenchymal stem cells for use with the invention may be derived from any human or non-human tissue source capable of providing a population of cells that produces a therapeutic effect when administered according to the methods disclosed herein. Suitable tissue sources include prenatal sources, postnatal sources, and combinations thereof. Tissues for deriving a suitable source of mesenchymal stem cells include, but are not limited to, bone marrow, blood (peripheral blood), dermis, periosteum, synovium, peripheral blood, skin, hair root, muscle, uterine endometrium, adipose, placenta, menstrual discharge, chorionic villus, amniotic fluid and umbilical cord blood. Mesechymal stem cells may be derived from these sources individually, or the sources may be combined (before or after enrichment) to produce a mixed population of mesenchymal stem cells from different tissue sources.

Mesenchymal stem cell compositions for use with the invention may comprise purified or non-purified mesenchymal stem cells. Purified mesenchymal stem cell compositions may be derived from clonal mesenchymal stem cells which have been expanded in culture. Alternatively, the purified composition may be derived from mesenchymal stem cells which have been isolated from a mixed population of cells such as a tissue preparation or a heterogeneous population of cells grown in culture. Methods for isolating mesenchymal cells from a mixed cell population are well known in the art and include, for example, FACS, cloning, density gradient centrifugation, magnetic sorting, affinity chromatography and serial passaging. Instructions for isolating mesenchymal stem cells suitable for practicing the invention are available to the skilled artisan and include, without limitation, the following: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No, 7,060,494; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705 (each incorporated by reference).

Mesenchymal stem cell compositions for practicing the invention may also comprise a non-purified (i.e. mixed) population cells. Such a mixed cell composition may be obtained by, for example, combining two or more different purified mesenchymal cell types, or by culturing a mixed population of cells which has been expanded from a tissue sample (i.e. a primary cell culture). It is also contemplated that the MSC composition of the invention may be derived from an enriched population of MSC obtained by the serial passage of a mixed population of cells such as a primary cell culture. Still further contemplated are mixed populations of cells obtained from combining an already mixed cell population with either one or more purified cell types, or a second mixed population of cells. Suitable methods for preparing a primary culture of mesenchymal cells are known in the art, and include, for example, those disclosed in Zhang et al. Cell Biol Int. November 2006 29, Sakaguchi et al. Arthritis Rheum. August 2005;52(8):2521-9, Izadpana et al. J Cell Biochem. December 1, 2006;99(5): 1285-97, Mareschi et al. J Cell Biochem. Mar. 1, 2006;97(4):744-54, and Pozzi et al. Exp Hematol. July 2006;34(7):934-42, each of which is incorporated by reference.

In addition to mixed populations, the MSC composition of the invention may comprise a purified population of cells. Such purified cells may be obtained from any source that provides a cell population having a therapeutic effect when administered according to the methods described herein. Without limitation, suitable purified mesenchymal stem cells for practicing the invention are disclosed in the following publications which are incorporated by reference: U.S. Pat. No. 5,654,186; U.S. Pat. No. 5,804,446; U.S. Pat. No. 5,980,887; U.S. Pat. No. 6,387,367; U.S. Pat. No. 6,541,249; U.S. Pat. No. 6,676,937; U.S. Pat. No. 6,852,537; U.S. Pat. No. 5,486,359; U.S. Pat. No. 7,056,738; U.S. Pat. No. 6,936,281; P.C.T. Pub. No. WO25112959; and U.S. Pat. Pub. No. 20060210544. As noted above, the purified cells referred to in these publications may be combined to form a non-purified composition of mesenchymal stem cells,

The cells that are used to make the MSC composition may have varying levels of plasticity. Thus, the composition may comprise multipotent MSCs capable of forming a plurality of cells of mesodermal lineage, or it may comprise precursor MSCs which are committed to the development of a particular specialized cell. In some embodiments of the invention, the MSC is comprised of endothelial precursor cells which have limited proliferative ability and which are committed to developing into an endothelial cell. Examples of endothelial precursor cells suitable for use with the invention include, but are not limited to, those listed in U.S. Pat. No. 5,980,887, U.S. Pat. No. 6,852,533, U.S. Pat. Pub. No. 20060210544 and U.S. Pat. Pub. No. 20060210544 (each incorporated by reference).

In some embodiments of the invention, the MSC composition is derived from pluripotent embryonic cells which have been induced to assume a mesenchymal phenotype in vitro. This may be achieved, for example, through the controlled introduction of mitogenic factors which influence the cells to differentiate along a mesodermal lineage. Suitable pluripotent embryonic cells for use with the invention are known in the art, and include, for example, the cells disclosed in U.S. Pat. No. 5,980,887 and U.S. Pat. Pub. No. 20060008902 which are incorporated by reference in their entirety.

In some embodiments of the invention, the MSG composition is made with cells which have, been transfected with a heterologous polynucleotide. The cells may be transfected with any protein that, when expressed, produces a therapeutic benefit to the CNS disorder the MSC composition is being used to treat. For example, the MSCs of the invention may be transfected to express an endothelial mitogen. An “endothelial cell mitogen,” as used herein, means any protein, polypeptide, mutein or portion that is capable of, directly or indirectly, inducing endothelial cell growth. Such proteins include, for example, acidic and basic fibroblast growth factors (aFGF and bFGF), vascular endothelial growth factor (VEGFX epidermal growth factor (EGF), transforming growth factor .alpha, and .beta. (TGF-.alpha. and TFG-.beta.) platelet-derived endothelial growth factor (PD-ECGF), platelet-derived growth factor (PDGF), tumor necrosis factor a (TNF-.alpha.), hepatocyte growth factor (HGF), insulin like growth factor (IGF), erythropoietin, colony stimulating factor (CSF), macrophage-CSF (M-CSF), granulocyte/macrophage CSF (GM-CSF) and nitric oxidesynthase (NOS). Suitable endothelial cell mitogens, and methods for their transfection, are disclosed in the following references which are incorporated by reference: Klagsbrun, et al., Annu. Rev. Physiol., 53:217-239 (1991); Folkman, et al., J. Biol. Chem., 267:10931-10934 (1992); Symes, et al., Current Opinion in Lipidology, 5:305-312 (1994); U.S. Pat. No. 5,332,671; and U.S. Pat. No. 5,980,887. Preferably, the endothelial cell mitogen contains a secretory signal sequence that facilitates secretion of the protein.

3. Treatment of CNS Disorders

The method of the invention may be used to treat any CNS disorder that is improved by administering (a) a neural stem cell composition to the neural side of a patient's blood brain barrier, and (b) a mesenchymal stem cell composition to the circulatory side of the patient's blood brain barrier.

CNS disorders treatable by the invention may be the result of a neurodegenerative disorder, ischemic disorder or neurological trauma (e.g. brain and spinal cord injuries). Examples of CNS disorders treated by the invention include, but are not limited to, ischemic stroke, hemorrhagic stroke, Parkinson's disease and Parkinsonian disorders, Huntington's disease, Alzheimer's disease, multiple sclerosis, amyotrophic lateral sclerosis, Shy-Drager syndrome, progressive supranuclear palsy; Lewy body disease, spinal ischemia, cerebral infarction, spinal cord injury, and cancer-related brain arid spinal cord multi-infarct dementia, geriatric dementia, cognition impairment, depression or traumatic injury, idiopathic orthostatic hypotension, progressive supranuclear palsy (Steele-Richardson-Olszewski syndrome), structural lesions of the cerebellum, such as those associated with infarcts, hemorrhages, or tumors, spinocerebellar degenerations such as those associated with Friedreich's ataxia, abetalipoproteinemia (e.g., Bassen-Kornzweig syndrome, vitamin E deficiency), Refsum's disease (phytanic acid storage disease), cerebellar ataxias, multiple systems atrophy (olivopontocerebell aratrophy), ataxia-telangiectasia, and mitochondrial multi system disorders, acute disseminated encephalomyelitis (postinfectious encephalomyelitis), adrenoleukodystrophy, adrenomyeloneuropathy, radiation-induced injury of the nervous system, chemotherapy-induced neuropathy (e.g., I encephalopathy), taxol neuropathy, vincristine neuropathy, diabetic neuropathy, autonomic, neuropathies, polyneuropathie, and mononeuropamies, and ischemic syridromes such as transient ischemic attacks, subclavian steal syndrome, drop attacks, and brain infarction.

3.1 Preparation of the Cell Compositions

Both the neural and mesenchymal stem cell compositions are made by suspending an appropriate amount of cells in a pharmaceutically acceptable carrier. As used herein the phrase “pharmaceutically acceptable” means the carrier, or vehicle, does not cause an adverse reaction when administered to a mammal. Such carriers are non-toxic and do not create an inflammatory or anergic response in the body. Pharmaceutically acceptable carriers for practicing the invention include any of the well known components useful for immunization such as, for example, culture media and phosphate buffered saline. Additional physiologically acceptable carriers and their formulations are well-known and generally described in, for example, Remington's Pharmaceutical Science (18.sup.th Ed., ed. Gennaro, Mack Publishing Co., Easton, Pa., 1990) and the Handbook of Pharmaceutical Excipients (4.sup.th ed., Ed. Rowe et al. Pharmaceutical Press, Washington, D.C.), each of which is incorporated by reference.

One aspect of the invention relates to the concentration of cells that is used in the neural and mesenchymal stem cell compositions. In this regard, the neural and mesenchymal stem cell compositions may be made using any cell concentration that provides a therapeutic effect when the compositions are administered according to the methods disclosed herein. Suitable concentrations for the neural and mesenchymal stem cell compositions may range between about 10⁴ to about 10⁷ cells/ml. The concentration of cells used for a particular treatment takes into consideration such factors as viscosity restrictions imposed by the diameter of the needle used for injection, as well as the volume of the compositions that are used for treatment. If more neural or mesenchymal stem cells are required than can be physically administered in a single injection, the invention contemplates simultaneous or sequential injections at the same or different injection sites.

3.2 Administration of the Cell Compositions

The neural stem cell composition may be administered (e.g. injected) to any site within the neural parenchyma (i.e. any region that is located on the neural side of the blood brain barrier of a subject). Accordingly, the neural stem cell composition may be administered to or near the brain, to or the near spinal cord, and combinations thereof.

In some embodiments of the invention, the neural stem cell composition is administered to the subject at least intrathecally. As used herein, the term “intrathecal administration,” or “intrathecally,” is intended to include delivering a neural stem cell composition directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, and U.S. Pat. No. 7,011,827, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of the neural stem cell composition to any of the above mentioned sites can be achieved by direct injection, or deposition, of the neural stem cell composition. The injection, or deposition, can be, for example, in the form of a bolus injection or continuous infusion of the neural stem cell composition.

In other embodiments of the invention, the neural stem cell composition is at least administered by injection into the brain, apposite the brain, and combinations thereof. The injection can be made, for example, through a burr hole made in the subject's skull. Suitable sites for administration of the neural stem cell composition to the brain include, but are not limited to, the cerebral ventricle, lateral ventricles, cisterna magna, putamen, nucleus basalis, hippocampus cortex, striatum, caudate regions of the brain and combinations thereof. The invention further contemplates administering the neural stem cell composition subdurally. Other modes of administration for the neural stem cell composition are known in the art, such as those disclosed in: Neural Transplantation: A Practical Approach, S. B. Dunnett & A. Bjorklund (Eds.) Irl Pr; (1992); Backlund, E.-O. et al., (1985) J. Neurosurg. 62:169 173; Lindvall, O. et al. (1987) Ann. Neurol. 22:457 468; and Madrazo, I. et al. (1987) New Engl. J. Med. 316:831 834 (each of which is incorporated by reference).

The mesenchymal stem cell composition is administered using any suitable method which introduces the composition to the circulatory side of a patient's blood brain barrier. This administration may take on a number of forms including, but not limited to, intravenous, intra-arterial, intramuscular, intraperitoneal, subcutaneous, intramuscular, intraabdominal, intraocular, retrobulbar and combinations thereof.

The invention contemplates varying the number of administrations of the neural and mesenchymal, stem cell compositions, as well as varying the intervals between these administrations. Thus, the number of administrations for one or both of the neural stem cell composition and the mesenchymal stem cell composition may range from a single administration, to at least 10 administrations. Multiple administrations for one or both of the stem cell compositions at different locations are also contemplated as part of the invention. The interval between the administration of the neural stem cell composition and the mesenchymal stem cell composition may range from no time at all (i.e. the simultaneous administration of the compositions) to at least one year. The stem cell compositions need not be administered in any particular order.

3.3 Ischemic Stroke

One non-limiting embodiment of the invention is a method for treating stroke comprising administering a neural stem cell composition that is derived from a primary culture of human fetal forebrain tissue, and a mesenchymal stem cell composition that is derived from human bone marrow. The neural stem cell composition is administered to the neural side of the blood brain barrier (i.e. the neural parenchyma) intrathecallly, while the mesenchymal stem cell composition is administered intravenously. The neural stem cell composition is made, for example, using about 100×10⁶ fetal neural stem in 2-4 ml of isotonic solution, while, the mesenchymal stem cell composition is made, for example, with about 50×10⁶ mesenchymal stem cells in 200 ml of isotonic solution. The administration of the cell compositions is carried out simultaneously, or sequentially.

This and other methods for treating stroke with the invention may administer another agent in combination with the neural and mesenchymal stem cell compositions. For example, agents such as endothelial cell mitogens which increase angiogenesis may be used. Some examples of suitable endothelial cell mitogens include, but are not limited to those disclosed in Pu, et al, Circulation, 88:208-215 (1993) (aFGF), Yanagisawa-Miwa, et al., supra (bFGF), Ferrara, et al., Biochem. Biophys. Res. Commun., 161:851-855 (1989) (VEGF), and Takeshita, et al., Circulation, 90:228-234 (1994).

### EXAMPLE 1

### Production of Neural Stem Cells

1. Donor Characteristic

The tissue donor of the neural tissue was tested for a variety of pathogens. PGR analysis for infection showed the mother's blood serum tested negative for the following infection markers: HIV-1 and -2; HPLV-I and II; HBV; HCV; CMV; HSV-1 and 2; toxoplasma gondii; mycoplasma; Epstein-Barr virus; ureaplasma; Chlamydia; and treporiema pallidum. Bacteriological tests showed the neural tissue was free of staphylococci, streptococci and neisseria gonorrhoeae.

2. Preparation of a Primary Cell Suspension of Neural Stem/Progenitor Cells (NSPC)

Source material for the neural stem cell suspension was the neocortical primordium from the brain of human fetuses at gestation weeks 9-11.

Initial Treatment of the Material

2.1. Using a quarantine workstation, either the entire forebrain or its fragments were isolated from the fetus with the use of ophthalmic forceps and the meninges were carefully removed.

2.2. The forebrain tissue was placed in a 30- or 60-mm plastic petri dish using forceps, and washed with Hank's solution, containing antibiotics (1 g of cefazolin and 250 mg of amphotericin B per 450 mL of solution), by adding 4-5 mL of the solution using a 5-mL plastic pipette.

2.3. The material was then washed with 10 mL of Versene solution for 1 minute.

2.4. The Versene solution was removed, 1 mL of growth medium was added, and the material was mechanically dissociated by repeated pipetting using a 5-mL plastic pipette or a Pipetman with a 1-mL tip until a single cell suspension was obtained.

2.5. The obtained cell suspension was transferred to a 15-mL Corning centrifuge tube, 10 mL of medium was added, and the suspension was pipetted.

2.6. The suspension was centrifuged for 5 minutes at 700 rpm; the supernatant was removed with a 10-mL plastic pipette.

2.7. The cell pellet was suspended in 2 or 5 mL of growth medium (depending on the amount of isolated cells) using a Pipetman. The live cells were counted with the use of a 35-mm petri dish, a Pipetman tip, and a Goryaev chamber. The number of live and dead cells in the suspension was counted by adding trypan blue to the selected sample. Material with a viability of at least 60% was regarded as suitable for culturing.

2.8. Cells were seeded using a Pipetman with a 1-mL tip in 2 or 5 mL of growth medium in petri dishes, 30 or 60 mL in diameter, depending on the amount of the obtained cells.

FIG. 1 shows a phase-contrast microscopy (PCM), primary suspension of dissociated brain tissue cells from an embryo at 10 weeks of development. Seeding density was 1-2 mL of cells per 1 mL of medium.

3. Growth Medium (Per 100 mL)

| | | Measurement | |
|---|---|---|---|
| Name | Amount | units | Manufacturer |
| F12 medium | 49 | mL | HyClone |
| DMEM medium | 49 | mL | HyClone |
| Gentamicin 4% | 250 | μL | Sigma |
| Glutamine | 2 | mM | PanEko |
| Fibroblast growth | 10 ng/mL | | ProSpec-Tany |
| factor | | | TechnoGene LTD |
| Epidermal growth | 10 ng/mL | | ProSpec-Tany |
| factor | | | TechnoGene LTD |
| Supplement N2 | 1 | mL | Gibco BRL |
| Heparin | 8 units/mL | | |
| FBS FetalClone III | 2 | mL | HyClone (SH3010903) |

4. Results of the Contamination Test

When negative results were obtained, the material was transferred from the quarantine workstation to the culturing workstation. If a positive result was obtained, the primary material was immediately destroyed and the workstation was sterilized.

5. Culturing and Characteristics of the Primary, Culture

5.1. Culturing was carried out under standard conditions: at 37° C. in an atmosphere of 5% CO₂.

5.2. The medium was replaced once every 3 days.

5.2.1. Spent medium was removed from the petri dish using a 10-mL pipette.

5.2.2. The same volume of fresh growth medium was added to the dish using a sterile 10-mL pipette.

5.3. Composition of the growth medium

| | | Measurement | |
|---|---|---|---|
| Name | Amount | units | Manufacturer |
| F12 medium | 49 | mL | HyClone |
| DMEM medium | 49 | mL | HyClone |
| Gentamicin 4% | 250 | μL | Sigma |
| Glutamine | 2 | mM | PanEko |
| Fibroblast growth | 10 ng/mL | | ProSpec-Tany |
| factor | | | TechnoGene LTD |
| Epidermal growth | 10 ng/mL | | ProSpec-Tany |
| factor | | | TechnoGene LTD |
| Supplement N2 | 1 | mL | Gibco BRL |
| Heparin | 8 units/mL | | |
| FBS FetalClone III | 2 | mL | HyClone (SH3010903) |

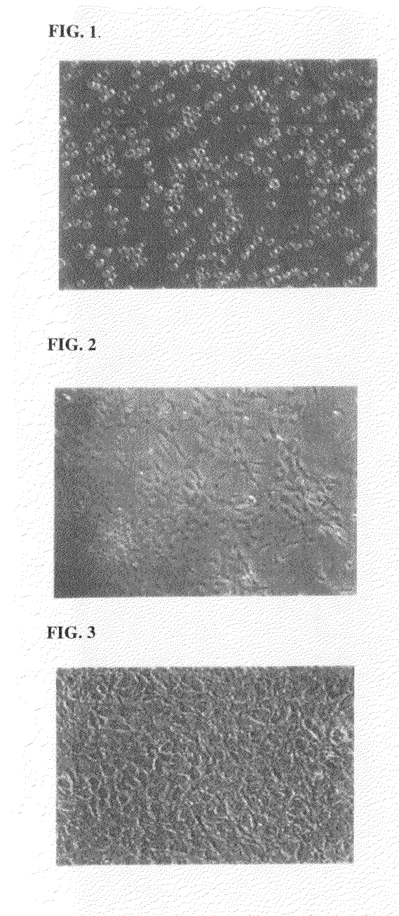

5.4. The time for obtaining a neuroectoderm cell culture ranged between 20 to 30 days. FIG. 2 shows neuroepithelial cells after 15 days in culture, while FIG. 3 shows neuroepithelial cells after 25 days in culture.

6. Culture Passaging

6.1. When cells reached confluence, the dishes with the cells were treated (washed) three times with Versene solution, then a trypsin solution (0.25%) was added to the dishes, and the dishes were left in an incubator for 3-5 minutes.

6.2. The sediment was resuspended and reseeded in new petri dishes or culture flasks at a 1:2 ratio in fresh growth medium.

7. Characteristics of the Passaged Culture

7.1. During culturing, the cultures were checked constantly and carefully in regard to bacteria and microscopic fungi, and also for the presence of bacteriological and viral infections. For this purpose, during passaging a portion of cells after passage 3-4 was given to the certification laboratory for PCR analysis. The sample tested negative for HBV, HCV, CMV, HSV-1 and 2, toxoplasma gondii, mycoplasma and Epstein-Barr virus.

7.2. Phenotyping of Passaged Cultures

7.2.1. Cytoflyorometric Analysis (FACS)

To perform the analysis, during passaging a portion of the cells was suspended in PBS, pH 7.4, after trypsinization at a concentration of 100,000 cells/mL, fixed in 1% methanol at 4° C. for 10 minutes, and then rinsed. Nonspecific binding was blocked by incubation in 1% BSA with 0.1% goatserum for 1 hour at room temperature. Then, the cells were rinsed in 3 volumes of PBS and centrifuged; the pellet was suspended in a 0.5% working solution of primary antibodies to 1% BSA with 0.1% goat serum. The cells were incubated for 40 minutes at 4° C. and rinsed with PBS. Mouse monoclonal antibodies (Chemicon or PharMingen) were also used. Nonspecific mouse (rabbit) IgG from the same companies was used as the negative control. Cells were incubated with antispecies antibodies, labeled with FITC or phycoerythrin, for 20 minutes, then rinsed with PBS, and analyzed with a flow cytofluorometer “FACS Calibur” (BD Biosciences). Results were analyzed using the program “MDI 2.8.”

| Neocr cells | % |
|---|---|
| R(av)2 | |
| HLA-DR | 2-10 |
| CD34 | 0.5-5 |
| CD45 | 0-1 |
| Nestin | 50-80 |
| Vimentin | 30-50 |
| b-Tubulin | 5-20 |
| GFAP | 5-10 |

7.2.2. Phase-Contrast Microscopy

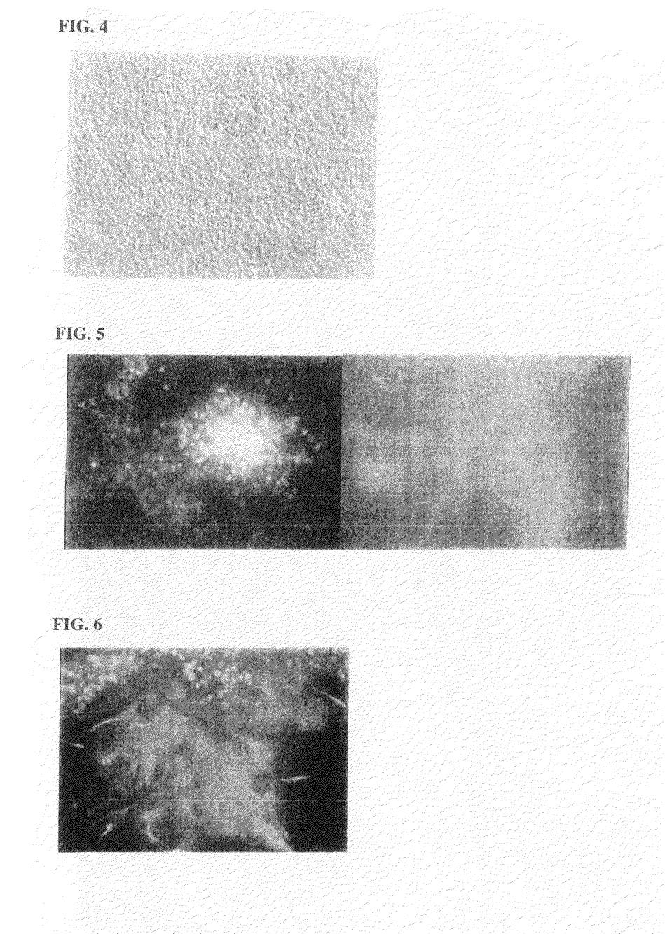

FIG. 4 depicts a phase contrast image of neuroepithelial cells after 5 passages.

7.2.3. Immunocytochemical Analysis

To phenotype the obtained cell culture, an immunohistochemical analysis was performed based on the expression of nestin, type IV protein (from intermediate neurofilaments, expressed in multipotent neuronal stem cells), beta-tubulin III (marker for early neuroblasts), and acid glial fibrillar protein (GFAP) (a marker for glioblasts and mature glial cells).

Immunohistochemical analysis was performed using, as primary antibodies, anti-nestin (1:10), anti acid glial fibrillar protein (1:400), anti beta-tubulin III (1:100). Secondary antibodies were anti-goat conjugated with phycoerythrin, and anti-goat conjugated with fluorescein isothiocyanate (FITC).

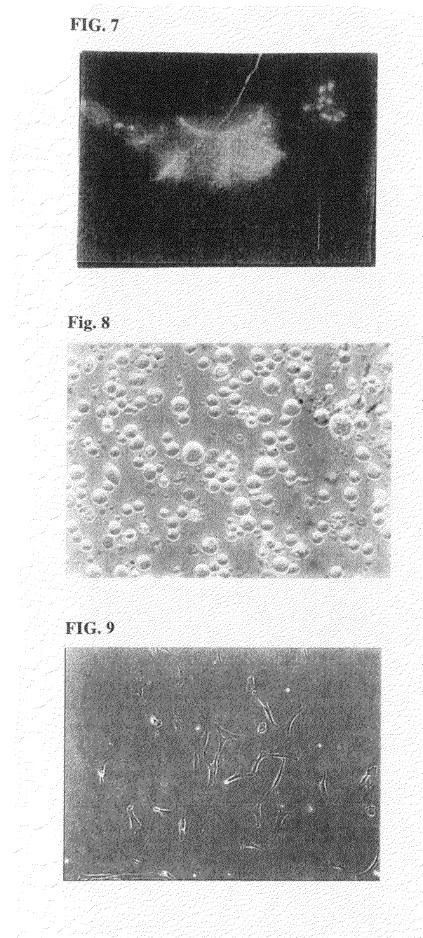

The neural stem cell culture showed expression of nestin (FIG. 5) and beta-tubulin III (FIG. 6) and GFAP (FIG. 7).

7.3. Cell Culturing Time

The number of passages depends on the state of the cells and their proliferative potential and is controlled by the expression of characteristic differentiation markers. The number of passages does not generally exceed 10 passages.

8. Cryopreservation

Before cryopreservation, a portion of the cells was used for a final contamination analysis and the rest of the cells were frozen.

8.1. The dishes or culture flasks with cells were treated (washed) three times with Versene solution, then a trypsin solution (0.25%) was added to the dishes, and the dishes were left in an incubator for 3-5 minutes.

8.2. The cells were carefully pipetted and the cell count was determined in a Goryaev chamber using a 35-mm petri dish and 1-mL tip for the Pipetman.

8.3. Cells were transferred by pipette to a 15-mL centrifuge tube and centrifuged for 6-7 minutes at 800-1000 rpm.

8.4. The supernatant was removed using a 10-mL pipette, and the cells were resuspended in the medium for freezing (human umbilical blood serum +7% dimethyl sulfoxide) at a concentration of 10 million cells per 1 mL of medium.

8.5. The cell suspension was transferred to 5-mL cryotubes using a 5-mL pipette.

8.6. The cryotubes were labeled according to the established standard.

8.7. The material was frozen to −80° C. in a programmable low-temperature freezer.

9. Characteristics of the Biotransplant

A freshly Obtained cell culture or a culture after its cryopreservation was used as the biotransplant.

9.1. Composition of a Biotransplant

The cell biotransplant was a sterile suspension of allogenic neural stem cell progenitors in physiological solution. The content of one flask of cell suspension is designated for only one patient and only for a single use. The amount of injected cells in a suspension and the volume were determined individually depending on specific objectives. The biotransplant was prepared for a specific patient 1-3 hours before the scheduled injection.

9.2. Amount and Percentage of Viable Cells

In preparing the biotransplant from a freshly prepared culture, the cell viability was at least 95%, which is checked with a test using trypan blue. In preparing a biotransplant from a cryopreserved culture, the cell viability after threefold rinsing was at least 90%.

### EXAMPLE 2

### Preparation of Mesenchymal Cells from Human Bone Marrow

1. Characteristics and Transport of the Donor Material

The source for the preparation of human MMSC was a bone marrow suspension (BMS) obtained by puncture of the iliac crest.

After the mandatory clinical, laboratory, and instrumental examinations of the patient (for autotransplantation) were performed, including:

1. The filling out of the medical history with an attachment of copies of all discharges from the medical history during previous stages of treatment and examination

2. Complete clinical blood tests

3. Complete blood biochemistry panel, with determination of renin, aldosterone, and brain natriuretic peptide

4. Blood group, Rhesus factor

5. Blood test for HIV and Wasserman test

6. Blood test for hepatitis B and C markers

7. Complete immune status

8. Chest x-ray

9. Ultrasound of abdominal organs, kidneys

10. ECG, Halter monitoring

11. Electrocardiography (rest, exercise)

12. Myocardial scintigraphy

13. Coronarography

BMS was harvested from the posterior iliac crest. The material was harvested in a procedures room with the necessary assortment of surgical and anesthesiology instruments. Exfusion of BMS was performed in accordance with the approved methodology, Instructions for the Preparation of Autologous Bone Marrow from Patients for Clinical Use—Ministry of Health, No. 14/2 of 8 Jan. 1980, and procedural recommendations “Transplantation of Bone Marrow in Acute Radiation Disease in Humans”—Ministry of Health of 3 Nov. 1986.

After the skin was treated with iodine-containing solutions, in the area of the posterior crest, a puncture was made through the skin and subcutaneous fatty tissue, through which aspiration needles were inserted. After this, the cortical plate of the iliac crest was pierced and the bone marrow was aspirated from the spongy substance of the bone. To collect 20-50 mL of bone marrow, several punctures of the bone cortical plate were made. This required the skin and subcutaneous fatty tissue to be moved aside using the aspiration needle. (The classic technology requires the aspiration of bone marrow in small batches from each puncture (3-5 mL in a 20-mL syringe); nevertheless, the amount of BMS, extracted from each puncture, can reach 20-50 mL, if the bone marrow flow is good.) Following aspiration, the bone marrow preparation was transferred to a polymer container with anticoagulant.

After harvesting was completed a bandage was applied where the skin was punctured and the BMS was immediately sent to the laboratory for further processing. The amount of BMS collected was 20-100 mL.

The BMS was transported to the laboratory in a sterile polymer container containing anticoagulant (heparin). Transportation of the BMS was carried out with strict observance of aseptic and temperature conditions: the container with the bone marrow suspension was placed in a hermetically sealing isothermal container for transport (+2 to +4° C.). Transporation of the BMS should not exceed 2 hours.

The bone marrow suspension received by the laboratory was tested for infectious agents (by PCR or serological/bacteriological tests). The sample was found to be negative for: HIV-1 and -2; HPV-I and II; HBV; HCV; CMV; HSV-1 and 2; toxoplasma gondii; mycoplasma; Epstein-Barr virus; ureaplasma; Chlamydia; treponema pallidum; enterococci; candida species; aspergillus species; e. coli; staphylococci; streptococci and neisseria gonorrhoeae.

The work with BMS in the laboratory is performed in accordance with the, recommendations “Instructions for Controlling the Sterility of Stored Blood, Its Components, Preparations for Preserved Bone Marrow, Blood Substitutes, and Preservation Solutions”—Ministry of Health No. 4-42-4-85 of 17 Sep. 1985.

In accordance with technological regulations, the cell phenotype is monitored for specific, satellite, and negative markers at all stages of the cell transplant preparation, and the contamination test is performed in accordance with the approved cell culture certificate.

2. Preparation of Fractions of Nucleated Cells from Human Bone Marrow Suspension

Plasma, extraneous material (bone fragments, fat), and erythrocytes were removed from the BMS aspirate.

2.1. An equal volume of PBS solution was added to the BMS aspirate.

2.2. The mixture was added over a Ficoll-Pague solution (Pharmacia) and centrifuged at 400 g for 30 minutes at 10° C.

2.3. The middle fraction of nucleated cells was collected, washed with PBS, and centrifuged at 200 g for 10 minutes.

2.4. The fraction was then resuspended in a hypotonic buffer solution for final elimination of erythrocytes and centrifuged. The hemoiyzed supernatant was removed.

The obtained suspension of nucleated cells was plated in plastic dishes in growth medium DMEM/F12 (1/1) (Gibco, Grand Island), containing 20% fetal calf serum (HyClone, USA), 2 mM glutamine, and antibiotics. The plating density of the primary cell suspension was 500,000-1,000,000 cells/cm² on average. Cells were cultured under standard conditions (at 37° C. in an atmosphere of 5% CO₂). After a day, unattached cells were removed, and attached cells were incubated to 70-80% confluence, which generally takes from 10 to 20 days. The culture medium was replaced every 3 days.

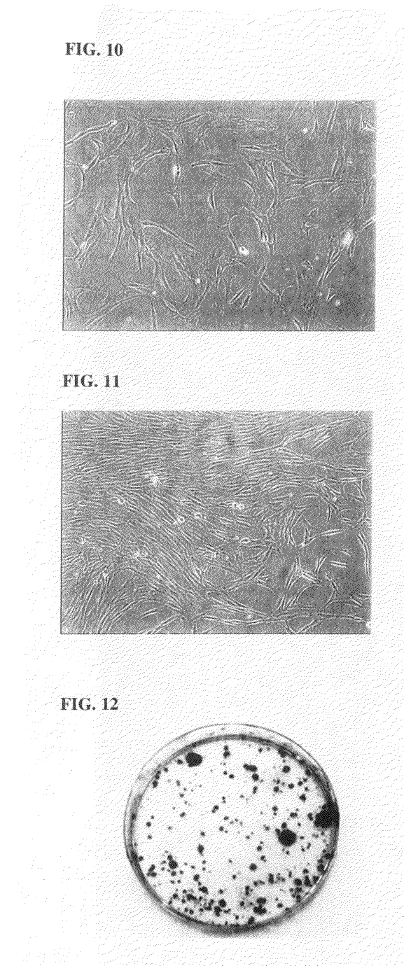

FIG. 8 is an image of the primary cell suspension obtained from the bone marrow aspirate, while FIGS. 9 and 10 depict the primary bone marrow cell culture at 4 and 10 days respectively.

After negative results on contamination were obtained, the material was transferred from the quarantine workstation to the culturing workstation.

3.3. Preparation of Cultures, Enriched with Multipotent Mesenchymal Stromal Cells (MMSC)

To select stem populations, MMSC cultures are serially cloned at a low density.

3.1. For this purpose, the condensed medium was removed from petri dishes with the primary monolayer culture, reaching 80% confluence, using a 10-mL sterile plastic pipette.

3.2. The petri dishes were washed three times with Versene solution using a 10-mL pipette; then using a 5-mL sterile plastic pipette 2-3 mL of trypsin solution (0.25%) was added, and the dishes were incubated at 37° C., 5% CO₂ for 5-7 minutes.

3.3. The suspension obtained after incubation was homogenized using a 10-mL sterile plastic pipette or Pipetman with a 1-mL tip.

3.4. Up to 10 mL of growth medium was added to the suspension and this was pipetted with a 10-mL sterile; plastic pipette until a homogeneous suspension was obtained.

3.5. The number of cells in the obtained suspension was counted using a Goryaev chamber.

3.6. The material was replated in new dishes at a density of 3-4 cells per 1 cm².

3.7. Up to 10 mL of growth medium was added to the petri dishes using a 10-mL sterile plastic pipette.

3.8. The medium was changed every 3 days.

Protocol for Replacing the Growth Medium (Once in 3 Days)

3.8.1, The condensed medium was removed from the petri dish with a 10-mL sterile plastic pipette.

3.8.2. The removed condensed,medium was replaced with new medium using a 10-mL sterile plastic pipette in an amount corresponding to the petri dish volume (90 mm-9-10 mL of medium).

3.9. After 10-14 days of culturing with monitoring with an inverted microscope, homogeneous, dense colonies of small cells (7-10 μm in diameter) with a large number of mitoses were selected from dishes, first treated with 1 mM EDTA.

3.10. The colonies were cultured further at a density of 10-50 cells per cm² in the same growth medium at 37° C. in an atmosphere, containing 5% CO₂ and at 95% humidity. The culture medium was replaced every 3 days.

3.11. To reach 50% confluence, the culture was plated at a plating density of 10-50 cells per cm². The number of culture passages did not exceed 5-7.

Protocol for Culture Passaging

3.11.1. The condensed medium was removed from petri dishes with the monolayer culture, reaching 50% confluence, using a 10-mL sterile plastic pipette.

3.11.2. 2-3 mL of trypsin solution was added to, the petri dishes using a 5-mL sterile plastic pipette, and the dishes were incubated at 37° C., 5% CO₂ for 5-7 minutes.

3.11.3. The suspension obtained after incubation was homogenized using a 10-mL sterile plastic pipette.

3.11.4. Up to 10 mL of nutrient medium was added to the suspension and this is pipetted with a 10-mL sterile plastic pipette until a homogeneous suspension was obtained.

3.11.5. The cells were counted using a Goryaev chamber.

3.11.6. The cells were then plated into new petri dishes plated at a density of 10-50 cells per cm² using a 10-mL sterile plastic pipette.

3.11.7. Medium was added to the, needed volume (to 9-10 mL) to petri dishes using a 10-mL sterile plastic pipette,

4. Growth medium Composition (Per 100 mL)

| | | Measurement | |
|---|---|---|---|
| Name | Amount | Units | Manufacturer |
| F12 medium | 49 | mL | HyClone |
| DMEM medium | 49 | mL | HyClone |
| Gentamicin 4% | 250 | μL | Sigma |
| Glutamine | 2 | mM | PanEko |
| Fibroblast growth factor | 10 ng/mL | | ProSpec-Tany |
| | | | TechnoGene LTD |
| Heparin | 8 U/mL | | |
| FBS FetalClone III | 15 | mL | HyClone |
| | | | (SH3010903) |
| Insulin | 1 μg | mL | |
| Transferrin | 10 μg | mL | |

5. Characteristics of the MMSC Culture

5.1. During culturing, cells were checked constantly and carefully in regard to bacteria and microscopic fungi, and also for the presence of bacteriological and viral infections. For this purpose, after the third passage, a portion of cells during passaging were given to the certified laboratory for analysis. PCR analysis showed the cells were negative for HBV, HCV, CMV, HSV-1 and -2, Toxoplasma gondii, Mycoplasma and Epstein-Barr virus.

5.2. Cell viability and morphology were assessed using a light microscope (FIG. 11).

5.3. Clonogenicity of the culture

After 10 days of culturing after low density plating (3-4 cells per 1 cm²), the colonies were counted in the control dish by staining with an alcohol solution of 0.5% crystal violet. FIG. 12 is an image; showing the colonies with positive staining.

5.4. Cytofluorometric Analysis (FACS)

To perform the analysis, during passaging a portion of the cells was suspended in PBS, pH 7.4, after trypsinization at a concentration of 100,000 cells/mL, fixed in 1% methanol at 4° C. for 10 minutes, and then washed. Nonspecific binding was blocked by incubation in 1% BSA and 0.1% goat serum for 1 hour at room temperature. Then, the cells were washed in three volumes of phosphate-buffered saline and centrifuged; the precipitate was suspended in a 0.5% working solution of primary antibodies to 1% BSA with 0.1% goat serum. After incubation for 40 minutes at 4° C., the cells were washed with phosphate-buffered saline, pH 7.4. Mouse monoclonal antibodies (McAb) to CD44, CD90, CD105, CD34, HLA ABC, and HLA DR purchased from PharMingen and Chemicon were used. Nonspecific mouse (rabbit) IgG from the same companies was used as the negative control. Incubation with anti-species antibodies, labeled with FITC or phycoerythrin was performed for 20 minutes. Cells were then washed in phosphate-buffered saline, pH 7.4, and analyzed in a volume of 1 mL in a flow cytofluorometer FACS Calibur (BD Biosciences). Results were analyzed using the program MDI 2.8.

Individual populations were identified in the flow cytofluorometer with use of the combination of McAb for differentiation and activation markers. The number of apoptotic cells was determined using McAb to CD95 (FAS/APO-1 antigen), and the number of hematopoietic cells using McAb to CD34. The functional activity of cellular immunity was evaluated based on the number of cells, expressing the receptor to IL2 (IL2R−CD3+, CD25+) and HLA-DR antigen on their surface, and also based on the number of activated cells (CD71+, CD38+) and activated NK (CD8+, CD16+).

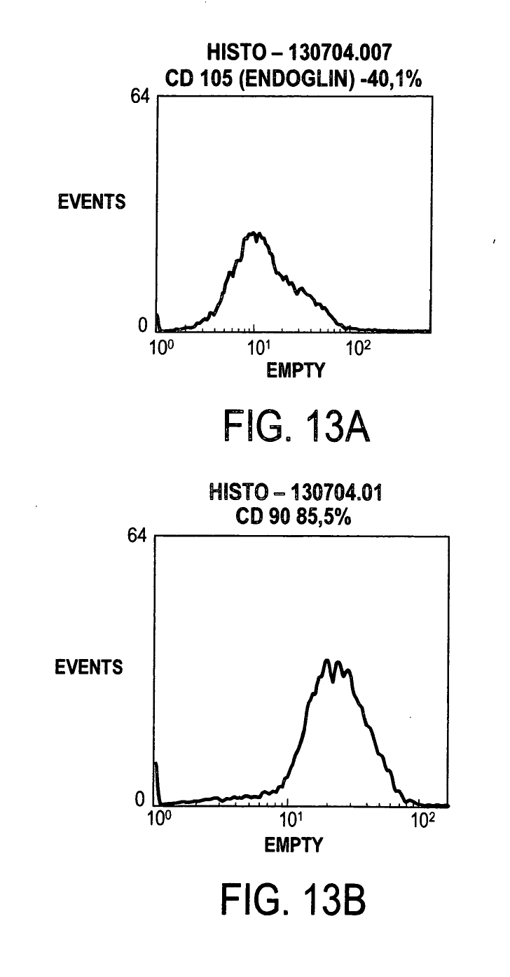

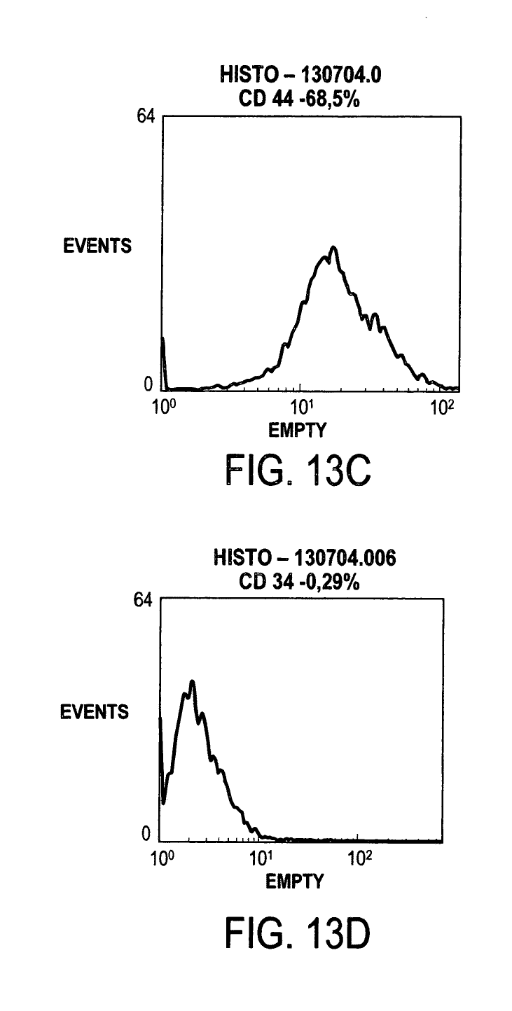

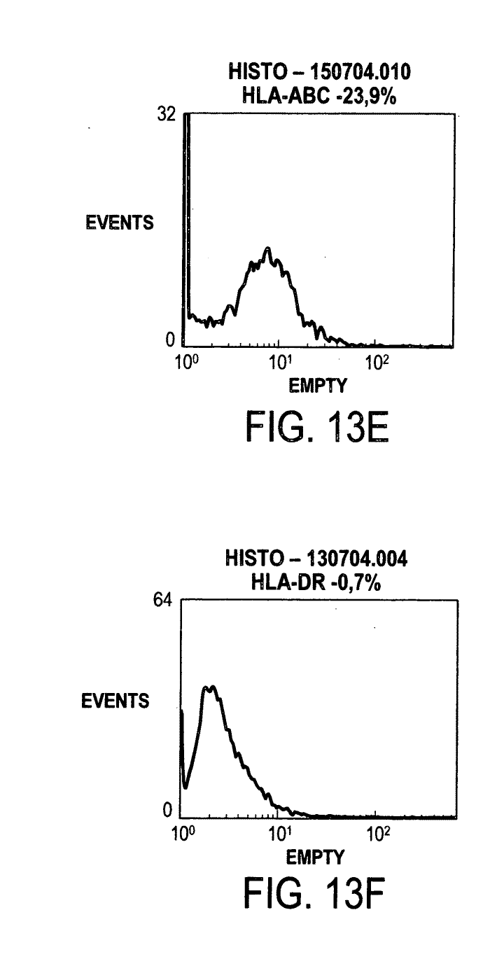

The primary marker of hematopoietic cells (CD34) and HLA DR in clonal cultures MMSC from bone marrow was expressed by less than 1% of cells (at the level of the negative control). The largest cell population (80-92%) was stained by antibodies to CD90 (80-95%), CD44 (60-75%), and endoglin CD105 (about 50%). Antigens MHC1 (HLA-ABC) were present on the surface of 5-30% of the cells. The fraction of positive cells changed minimally during passaging, but remained unvaryingly low overall (see FIGS. 13a-f).

6. Cryopreservation

Before cryopreservation, a portion of the cells was used for the contamination test (final infection check), and the rest were frozen.

6.1. The condensed medium was removed from petri dishes, with the confluent monolayer culture, using a 10-mL sterile plastic pipette.

6.2. The cell culture was washed three times with Versene solution using a 10-mL sterile pipette.

6.3. 2 mL of trypsin solution was added to a petri dish using a 10-mL sterile plastic pipette, and the dish was incubated at 37° C., 5% CO₂ for 10 minutes.

6.4. The suspension obtained after incubation was homogenized using a 10-mL sterile plastic pipette.

6.5. Up to 5 mL of nutrient medium was added to the suspension using a 10-mL sterile plastic pipette and the suspension was homogenized using a 10-mL sterile plastic pipette.

6.6. The cells were counted using a Goryaev chamber.

6.7. The suspension was transferred to a 15-mL centrifuge tube using a 10-mL sterile pipette and diluted to 10 mL with Hank's solution using a 10-mL sterile pipette.

6.8. The suspension was centrifuged for 10 minutes at 1000 rpm.

6.9. The supernatant was removed using a 10-mL pipette, and the cells were resuspended in the medium for freezing (human umbilical blood serum +7% dimethyl sulfoxide) at a concentration of 10 million cells per 1 mL of the medium for freezing.

6.10. The cell suspension was transferred to 5-mL cryotubes using a 5-mL pipette.

6.11. The cryotubes were labeled according to the established standard.

6.12. The material was frozen to −80° C. in a programmable low-temperature freezer.

7. Characteristics of the Biotransplant

A freshly obtained cell culture or a culture after its cryopreservation was used as the biotransplant.

7.1. Composition of a biotransplant

The cell biotransplant was a sterile suspension of autologous or allogenic mesenchymal stem cells, resembling fibroblasts, in physiological solution. The content of one flask of cell suspension was designated for only one patient and only for a single use. The amount of injected cells in a suspension and the volume were determined individually depending on specific objectives. The biotransplant was prepared for a specific patient 1-3 hours before the scheduled injection.

7.2. Amount and fraction of viable cells

In preparing the biotransplant from a freshly prepared culture* the cell viability was at least 95%, which was checked with a test using trypan blue. In preparing a biotransplant from a cryopreserved culture, the cell viability after threefold rinsing was at least 90%.,

### EXAMPLE 3

### Treatment of Isochemic Stroke in Humans

3.1 Inclusion Criteria

Patients with a known motor defect (such as heniparesis) following a completed ischemic or hemorrhagic cerebral infarction.

Neuroanatomical relationship between neurological deficit and imaging defined stroke (cerebral CT scan)

No substantial change in neurological deficit for two months before enrollment, per medical history.

Time interval between one and six years from any documented stroke.

For women of childbearing age, a negative pregnancy test within 2 weeks before cell transplantation and a willingness to practice adequate contraception for one year post implantation of stem cells.

Ages 18 to 80 of both genders.

Able to comprehend the investigational nature of the study and provide informed consent.

Provide initial event (historical medical data) and current imaging studies as well as current neurological assessment as determined by medical staff.

Willing to comply with study protocols i.e. after stem cell treatment, speech and physical therapy.

Normal blood counts including liver and kidney function tests.

3.2 Exclusion Criteria

Active infection: Syphilis, Hepatitis C or HIV

Autoimmune disease, rheumatoid arthritis, systemic lupus.

History of active cancer within the past 5 years, including all brain cancers, and excluding skin cancers (not in remission).

History of blood disorders.

Pregnant or lactating. Patients are requested to use effective birth control for one year after last stem cell treatment.

Severe psychiatric illness, mental deficiency sufficiently severe as to make informed consent impossible.

3.3 Cell Injections

A single neural stem cell injection, over a one minute period, was performed using 100×10⁶ neural stem cells suspended in 2-4 ml of isotonic solution. Neural stem cells were injected intralumbarly between lumbar vertebrae L3 and L4.

Mesenchymal stem cells were prepared for injection by suspending 50×10⁶ mesenchymal stem cells in 200 ml of isotonic solution. Mesenchymal stem cells were administered intravenously in a single injection over a 40-60 minute period. The mesenchymal stem cells were administered approximately 15 minutes after the injection of the neural stem cells.

3.3 Secondary Treatment

To support the viability and growth of the transplanted cells, two secondary treatments were administered: (1) 2.0 ml of a B-vitamin complex (100 mg B1, 100 mg B6 and 1 mg B12) was administered intramuscularly; and (2) 500 ml of an aminoplasmal solution was administered by intravenous drip. The aminoplasmal solution contained the following:

| | |
|---|---|
| Isoleucine | 0.51 g/100 ml |
| Leucine | 0.89 g/100 ml |
| Lysine | 0.56 g/100 ml |
| Methionine | 0.38 g/100 ml |
| Threonine | 0.41 g/100 ml |
| Valine | 0.48 g/100 ml |
| Arginine | 0.92 g/100 ml |
| Histidine | 0.52 g/100 ml |
| Alanine | 1.37 g/100 ml |
| Glycine | 0.79 g/100 ml |
| Aspartic Acid | 0.13 g/100 ml |
| Asparagine | 0.327 g/100 ml |
| Glutamic Acid | 0.46 g/100 ml |
| Ornithine | 0.25 g/100 ml |
| Phenylalanine | 0.51 g/100 ml |
| Tryptophan | 0.18 g/100 ml |
| Proline | 0.89 g/100 ml |
| Serine | 0.24 g/100 ml |
| Tyrosine | 0.10 g/100 ml |

3.4 Follow-Up Evaluations and Results

Follow up evaluations were performed at two and six month intervals for up to 2 years after the start of the study. Neurological, physical therapy assessment and speech assessment were evaluated. Activities of daily living and neurological questionnaire tools were utilized, standardized for easy assessment of patients' progress as well as individualized to the patient's neurological problem. Emphasis was placed on physical, mental, neurological functioning and activities of daily living improvements as benchmarks for assessment of improvement.

Some patients were evaluated using pre- and post-pet scans but radiological assessments were not used in the investigation.

Adverse stem cell experiences were, graded according to the NCI (National Cancer Institute) Common Toxicity Criteria Version 2.

Clinical adverse, events not classified by this scale were categorized using the following definitions:
1. MILD—discomfort noted, but no disruption of baseline daily activity
2. MODERATE—discomfort noted of sufficient severity to reduce or adversely
3. SEVERE—incapacitating, with inability to work or perform normal daily activities

Clinical Observations

| Patient | Condition | Treatment Date | Pre Treatment | Post Treatment |
|---|---|---|---|---|
| Female (Age | Alzheimer's | January 2006 | Forgetfulness | Quality of life returned |
| 75) | | | Disorientation | Able to engage in household |
| 6032.0126.0000 | | | Physically and verbally | chores such as washing clothes, |
| | | | aggressive | cleaning house, cooking and |
| | | | Didn't recognize family | gardening |
| | | | members | Can recall detailed information |
| | | | Neglected appearance | about her past and her family |
| | | | Inability to respond to questions | Follows directions well |
| | | | quickly or effectively | No longer aggressive |
| | | | Didn't know where she lived, | Can complete complex thought |
| | | | date, year | processes when evaluated by |
| | | | Could not remember her past | neurologist - such processes that |
| | | | Thought she was in a psychiatric | require active imagination to solve |
| | | | clinic | problems |
| | | | | Appearance vastly improved - |
| | | | | cares, about her appearance |
| Female (Age | Spinal Cord | April 2006 | Unable to stand/Balance | Standing/Balancing unassisted |
| 60) | | | Dependent on others for bathing, | Walking with walker |
| 6001.1903.0000 | | | eating, dressing | Cooking/preparing food |
| | | | Could not prepare meals | Brushing Hair |
| | | | Could not groom herself | Writing |
| | | | Not ambulatory | Using Computer |
| | | | Loss of independence | Has experienced sensation in legs |
| | | | No strength in arms, hands, legs | Driving vehicle with hand controls |
| | | | No feeling in legs | Quality of life vastly improved |
| | | | | Regained a good portion of her |
| | | | | self sufficiency and independence |
| | | | | Continues to improve |
| Male (Age 89) | Alzheimer's | June 2006 | Very advanced case of | Sleeping better and through the |
| 6007.0126.0000 | | | Alzheimer's (+10 years) | night |
| | | | Extreme forgetfulness | Engages in life activities again |
| | | | Disorientation | such as pruning trees, watering the |
| | | | Could not answer questions | yard, reading the paper and doing |
| | | | accurately about his past, dates, | crossword puzzles |
| | | | etc. | Memory improved, can remember |
| | | | Unable to sleep through the | some events where before he could |
| | | | night | not |
| | | | Required constant supervision | Family reports him to be much |
| | | | Uninterested and unengaged in | happier and interactive |
| | | | daily living - slept a lot | |
| Male (Age 69) | Stroke | June 2006 | Contracture of right arm | Contracture release |
| 6021.1920.0000 | | | Constant drooling of the mouth | Regained use of left leg/arm |
| | | | Left side of face paralyzed | Can draw and write as before |
| | | | Left side of body, arm, leg, | stroke |
| | | | numb and unable to be used | Sees and draws on both sides of |
| | | | Minimal ability to walk | the page |
| | | | Unable to get up from the floor | Can see food on both sides of the |
| | | | or a sitting position unassisted | plate and can eat unassisted |
| | | | Required assistance with | Speech returned and is as fluid as |
| | | | bathing, eating, hygiene | before stroke |
| | | | Could only see food on one side | Able to swim fist time since |
| | | | of the plate and would only eat | stroke |
| | | | on that side | Able to walk and stand for |
| | | | Unable to read or concentrate on | extended periods of time |
| | | | things for more than a matter of | unassisted |
| | | | minutes and only with excessive | Can place weight on his left leg |
| | | | drooling | and can do knee bends |
| | | | Speech markedly impaired and | Can hop on the left leg |
| | | | unable to respond quickly or | Drooling completely stopped |
| | | | effectively to others verbally | Strength markedly increased |
| | | | | (See Video - Sachio Nishisako) |
| Male (Age 71) | Stroke | August 2006 | Slouched posture | Standing upright |
| 6010.1920.0000 | | | Very tight contracture on arm | Contracture release |
| | | | Walking markedly impaired with | Improved speech - occasionally up |
| | | | left leg dragging a bit | to 5 word sentences |
| | | | Speech limited to a few words | Strength much improved and |
| | | | and could only say one word at a | endurance improved |
| | | | time | Lucidity increase |
| | | | Very weak muscles in arms/ | Activities of Daily Living |
| | | | legs | improved and increased |
| Male (Age 83) | Alzheimer's | November 2006 | Very advanced case of | Sleeps through the night - not |
| 6008.0126.0000 | | | Alzheimer's (+7 years) | roaming in the middle of the night |
| | | | Extreme forgetfulness | Memory markedly improved |
| | | | Extreme disorientation | (remembered Doctor when he |
| | | | Could not answer questions | called after not seeing him for 2 |
| | | | accurately about his past, dates, | months) |
| | | | etc. | Activities of daily living increased |
| | | | Unable to sleep through the | and improved per wife |
| | | | night and would wander | |
| | | | confused through the house | |
| | | | Required constant supervision | |
| Female (Age | Stroke | November 2006 | Markedly impaired speech | Speech has improved and |
| 65) | | | Contracture of arm | continues to improve with speech |
| 6016.1920.0000 | | | Mobility limited by damage to | therapy |
| | | | left side of the body | Strength has increased |
| | | | | Contracture release |
| Male (Age 54) | Parkinson's | November 2006 | Mid-stage Parkinson's with | Immediate post treatment results |
| 6018.1611.0000 | | | marked tremor | have significantly reduced tremor |

I claim:

1. A method for treating a CNS disorder in a subject having a blood brain barrier (BBB), said method comprising the steps of:
administering on the neural side of said BBB a composition comprising a therapeutically effective amount of neural stem cells; and
administering on the circulatory side of said BBB a composition comprising a therapeutically effective amount of mesenchymal stem cells.

2. The method of claim 1, wherein said neural stem cells comprise a substantially purified population of neural stem cells.

3. The method of claim 1, wherein said neural stem cells comprise-a mixed population of neural stem cells.

4. The method of claim 3, wherein said mixed population of neural stem cells comprises a combination of two or more, purified populations of neural stem cells.

5. The method of claim 2, 3 or 4, wherein said neural stem cells comprise fetal neural stem cells.

6. The method of claim 1, wherein said mesenchymal stem cells comprise a substantially purified population of mesenchymal stem cells.

7. The method of claim 1, wherein said mesenchymal stem cells comprise a mixed population of mesenchymal stem cells.

8. The method of claim 7, wherein said mixed population of mesenchymal stem cells comprises a combination of two or more purified populations of mesenchymal stem cells.

9. The method of claim 1, wherein said composition comprising neural stem cells comprises (a) a substantially purified population of neural stem cells, (b) a mixed population of neural stem cells, or (c) a combination of (a) and (b).

10. The method of claim 9, wherein said composition of neural stem cells comprises prenatal neural stem cells, post-natal neural stem cells, or a combination thereof.

11. The method of claim 1, wherein said composition comprising mesenchymal stem cells is selected from (a) a purified mesenchymal stem cell population, (b) a mixed population of mesenchymal stem cells, or (c) a combination of (a) and (b).

12. The method of claim 11, wherein said composition of mesenchymal stem cells comprises prenatal mesenchymal stem cells, post-natal mesenchymal stem cells, or a combination thereof.

13. The method of claim 1, wherein said neural side of the BBB is intrathecal or intracranial.

14. The method of claim 1, wherein said circulatory side of the BBB is intravenous.

15. The method of claim 1, wherein said step of administering on the neural side of the BBB and said step of administering on the circulatory side of the BBB are performed simultaneously.

16. The method of claim 1, wherein said step of administering on the neural side of the BBB and said step, of administering on the circulatory side of the BBB are performed sequentially.

17. The method of claim 16, wherein the duration between said sequential steps is less than a year.

18. The method of claim 1, wherein said GNS disorder is selected from hemorrhagic stroke, multiple sclerosis, cerebral palsy, Parkinson's disease, Alzheimer's disease, ischemic injury, and traumatic injury.

19. The method of claim 1, wherein said mesenchymal stem cells are derived from one or more sources consisting of bone marrow, umbilical cord blood, peripheral blood, placenta, chorionic villus, adipose tissue, menstrual discharge, and amniotic fluid.

20. The method of claim 1, wherein said neural stem cells are derived from one or more sources consisting of central nervous system tissue, bone marrow, umbilical cord blood, peripheral blood, placenta, chorionic villus, and amniotic fluid.
